(19) United States
(12) Patent Application Publication
(10) Pub. No.: US 2007/0123984 A1
(43) Pub. Date: May 31, 2007

(54) Ligament attachment and repair device

(75) Inventor: Robert A. Hodorek, Warsaw, IN (US)

Correspondence Address: BAKER & DANIELS LLP;111 E. WAYNE STREET, SUITE 800, FORT WAYNE, IN, 46802, US

(73) Assignee: Zimmer Technology, Inc.

(21) Appl. No.: 11/259,003

(22) Filed: Oct. 26, 2005

(51) Int. Cl. A61F 2/08

(52) U.S. Cl. 623/13.14

(57) ABSTRACT

An implantable attachment device for attaching an end of a ligament to a bone. The attachment device includes a patch formed of a woven material. The patch includes a bone engaging face and a ligament engaging face. The patch is positionable between the bone and the end of the ligament and is adapted to be fastened to a surface of the bone such that the bone engaging face bears against the surface of the bone. The ligament engaging face is adapted to receive a ligament fastener for attaching the end of the ligament to the patch. The device may also include a reinforcing patch positionable atop and attachable to the patch, such that the end of the ligament is positioned between the ligament engaging faces of the patch and the reinforcing patch.

### BACKGROUND

The present invention relates to devices for attaching an end of a ligament to a bone or for repairing a tear in a ligament.

Injury or damage to a ligament may cause an end of the ligament to detach from the bone. In these cases, it is desirable to re-attach the end of the ligament to the bone. In other cases, injury or damage to the ligament may cause a centralized tear in the ligament at a position between the two ends, leaving the two ends attached to the bone. If the ligament is salvageable, it is desirable to reunite the two loose ends of the torn ligament. In either of these cases, if the ligament is not salvageable, the ligament may be removed and an artificial or harvested graft ligament may be implanted. Many current ligament attachment systems require, first, drilling a tunnel into the bone. The ligament is attached to the bone by using a fastener to draw the ligament into and secure the ligament in the bone tunnel. Although effective, this system may be complex and the procedure may be significantly invasive.

There is a need for a relatively simple and minimally invasive implantable attachment device for attaching an end of a ligament to a bone or for repairing a tear in a ligament.

### SUMMARY

The present invention provides an implantable attachment device for attaching an end of a ligament to a bone. In one form, the attachment device includes a patch. The patch includes a bone engaging face and a ligament engaging face. The patch is positionable between the bone and the end of the ligament and is adapted to be fastened to a surface of the bone such that the bone engaging face bears against the surface of the bone. The ligament engaging face is adapted to receive a ligament fastener for attaching the end of the ligament to the patch. The device may also include a reinforcing patch positionable atop and attachable to the patch, such that the end of the ligament is positioned between the ligament engaging faces of the patch and the reinforcing patch.

The present invention also provides an implantable device for re-attaching two ends of a torn ligament to one another. In one form, the device includes a patch having an exterior face and an interior face. The patch is adapted to receive at least one ligament fastener for attaching the two ends of the ligament to the interior surface of the patch. The patch is formable to wrap around the two ends of the ligament.

In another form, the device includes a first patch and a second patch. Each of the first and second patches may be formed of a woven material and includes an interior ligament face and an exterior face. The woven material is adapted to receive a ligament fastener for attaching the ends of the ligament to the interior ligament face. The first patch is positionable adjacent a first side of the ends of the ligament. The second patch is positionable atop the first patch and adjacent an opposite second side of the ends of the ligament such that the ends of the ligament are positioned between the interior faces of the first and second patches. The second patch is attachable to the first patch.

In still another form, the invention contemplates a ligament repair system for replacing a damaged ligament. The ligament replacement system includes a replacement ligament having a first bone engaging end and a second bone engaging end. First and second patches are respectively coupled to the first and second bone engaging ends, and each of the first and second patches has a bone engaging face and a ligament engaging face. Each of the first and second patches is adapted to be fastened to a surface of a respective bone such that the bone engaging face bears against the surface of the respective bone. The system also includes a ligament fastener. The ligament engaging face is adapted to receive the ligament fastener to attach the first and second bone engaging ends of the ligament to the first and second patches, respectively.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is a medial aspect of a knee with a ligament attached at one end to the tibia using an attachment device of the present invention;

FIG. 2A is a front view of the attachment device of FIG. 1;

FIG. 2B is a side view of the attachment device of FIG. 1;

FIG. 3 is a medial aspect of a knee with the respective ends of a ligament attached to the tibia and femur using an attachment device of the present invention;

FIG. 4A is a medial aspect of a knee with a ligament attached to the tibia at one end using an attachment device of the present invention;

FIG. 4B is an anterior aspect of the knee and the attachment device of FIG. 4A;

FIGS. 5A-5D illustrate a medial aspect of a knee with a ligament having a centralized tear and the repair of that tear using an attachment device of the present invention;

FIG. 6A is an interior surface view of an attachment device of the present invention;

FIG. 6B is an interior surface view of the attachment device of FIG. 6A in a partially folded configuration;

FIG. 6C is a perspective view of the attachment device of FIG. 6A in a substantially folded configuration; and

FIG. 6D is a medial aspect of a knee wherein the attachment device of FIG. 6A is used to repair a ligament.

Corresponding reference characters indicate corresponding parts throughout the several views. Although the drawings represent embodiments of the present invention, the drawings are not necessarily to scale and certain features may be exaggerated in order to better illustrate and explain the present invention. Although the exemplification set out herein illustrates embodiments of the invention, in several forms, the embodiments disclosed below are not intended to be exhaustive or to be construed as limiting the scope of the invention to the precise forms disclosed.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

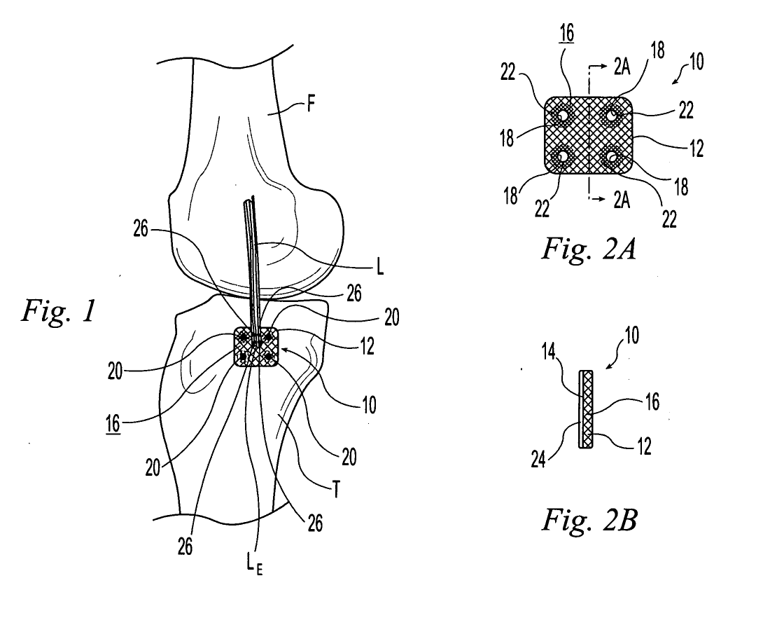

Referring first to FIG. 1, a knee joint is illustrated including femur F, tibia T and tibial collateral ligament L. Ligament L, due to damage or injury to the knee, has detached from tibia T at ligament end L_{E}. Attachment device 10 is used to re-attach ligament end L_{E} to tibia T. Referring now to FIGS. 1, 2A and 2B, device 10 includes patch 12 having bone engaging face 14 and ligament engaging face 16. As is described in further detail below, bone engaging face 14 is adapted to bear against a bone, such as tibia T, while ligament engaging face 16 is adapted to receive ligament fastener 26 for fastening ligament end L_{E} to patch 12.

Patch 12 may be formed of any material that would permit attachment of ligament fastener 26 thereto. For instance, patch 12 may be formed of a woven material. The woven material may be woven of any surgical grade fibers including metal fibers, plastic fibers, synthetic fibers such as Kevlar®, rayon, acetate, and polyester or mixtures thereof. Alternatively, the material of patch 12 may be in the form of a porous fabric or a molded lattice. The composition and structure of the material of patch 12 is such that it provides patch 12 with a strength and rigidity that resists deformation and tearing when attached ligament L pulls on patch 12 during knee movement. At the same time, the composition and weave of patch 12 may maintain some flexibility to allow patch 12 to form to the surface of tibia 12. Patch may have a variety of shapes and thicknesses. For instance, patch 12 may be substantially flat, thin and square. Alternatively, patch 12 may be elongated to provide more ligament contact area. As discussed below, patch 12 may be contoured on one or both sides to facilitate attachment to tibia T and attachment of ligament end L_{E} thereto.

Patch 12 is adapted to be fastened to tibia T using bone fasteners 20. For example, if patch 12 is formed of a woven material, the material may be woven loosely enough to allow a driving end of bone fasteners 20 to be driven through patch 12, yet woven tightly enough to prevent the head of fasteners 20 from passing through the material thereby securing patch 12 to tibia T. Alternatively, as illustrated in FIGS. 1 and 2A, patch 12 may include bone fastener openings 18 extending therethrough from ligament engaging face 16 to bone engaging face 14. Bone fastener openings 18 receive bone fasteners 20 to secure patch 12 to tibia T. Bone fasteners 20 may be any fasteners capable of attaching to bone including screws, pins, nails, anchors, and staples. Patch 12 may be reinforced about the perimeter of openings 18 to provide additional support, durability and resistance to tearing around fasteners 20. For instance, the woven material forming patch 12 may be more tightly woven around openings 18 to create stronger reinforced area 22 surrounding openings 18. Alternatively, patch 12 may include grommets around openings 18.

As mentioned above, ligament engaging face 16 is adapted to receive ligament fastener 26 for attaching ligament end L_{E} to patch 12. Referring to FIG. 1, the material of patch 12 is accessible at ligament engaging face 16 and, as noted above, is structured to allow the fixation of ligament fastener 26 thereto. For instance, ligament fasteners 26 may be sutures formed of surgical thread or fibers that may be securely stitched to the material of patch 12. Alternatively, ligament fasteners may be in the form of staples or clips that can be securely fastened to the material of patch 12. To further facilitate the attachment of ligament L_{E} to patch 12, ligament engaging face 16 may be contoured to receive ligament end L_{E} and to allow ligament end L_{E} to rest in and against ligament engaging face 16. For instance, ligament engaging face 16 may have a recess defined therein sized and configured to receive ligament end L_{E}.

Also noted above, bone engaging face 14 is adapted to bear against tibia T. More particularly, bone engaging face 14 may be contoured to complement the surface of tibia T. Alternatively, patch 12 may be flexible such that bone engaging face 14 conforms to the surface of tibia T. In another alternative, bone engaging face 14 may be structured to provide a pliable layer that is capable of conforming to the surface of tibia T. Additionally or alternatively, the material of patch 12 may be structured to provide bone engaging face 14 with a mesh that facilitates the ingrowth of bone into patch 12, thereby further securing patch 12 to tibia T.

Further, as illustrated in FIG. 2B, bone engaging face 14 may include bone engaging layer or coating 24 having a composition designed to facilitate the engagement of bone engaging face 14 to tibia T. For example, bone engaging layer 24 may provide a porous scaffolding for the ingrowth of tibia T. Such porous scaffolding may be formed of hydrogels. Suitable hydrogels may include polyvinyl pyrollidone, polyvinyl alcohol, polyurethane, polyacrylamide, calcium alginate, hyaluronic acid, polyethylene glycol and polyhydroxyethylmethacrylate. In addition or in the alternative, bone engaging layer 24 may include biologic material such as pharmaceuticals and therapeutics. For example, bone engaging layer 24 may include pharmaceuticals such as antibiotics, anticoagulants, anti-inflammatories, steroids and other compositions that prevent infection, facilitate healing and/or encourage bone ingrowth. Bone engaging layer may also include therapeutics such as growth factors, periosteal cells, ligament cells, blood, antibodies or other materials. Such pharmaceuticals and therapeutics may be loaded into a hydrogel scaffold or may be suspended in a composition, such as hydrogel, and formed into layer 24.

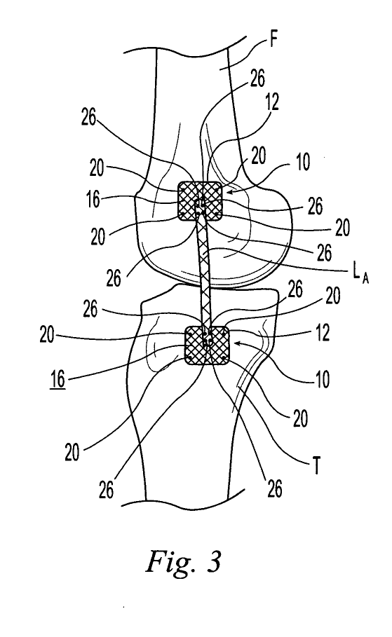

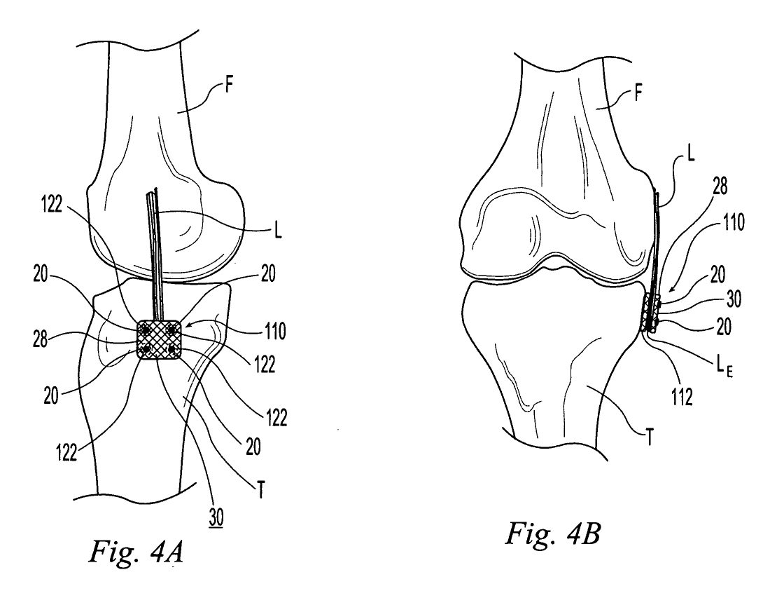

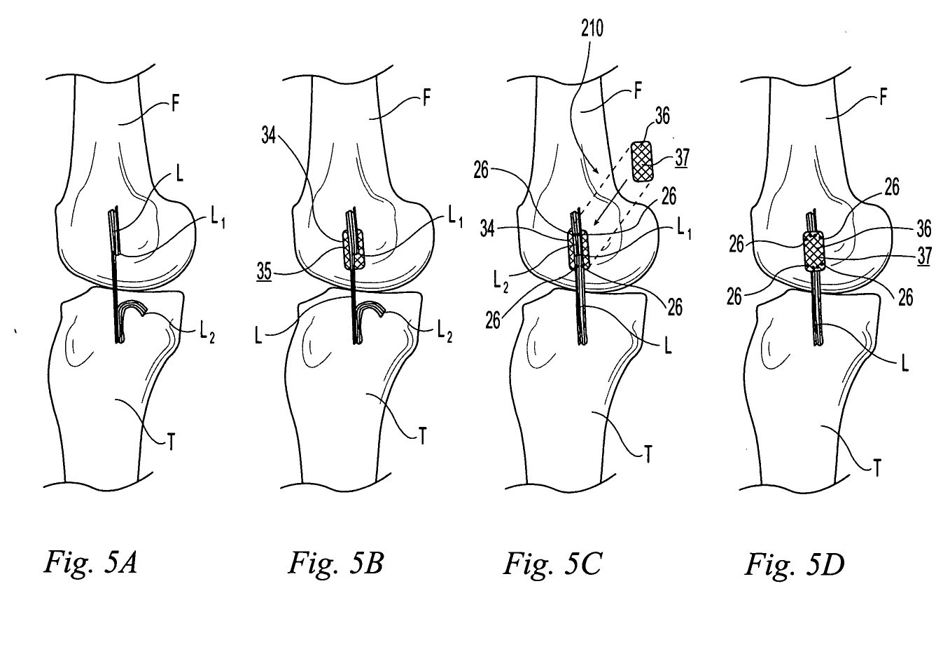

Turning now to FIG. 3, device 10 may be used to attach both ends of a ligament to any bone. Furthermore, device 10 is not restricted to re-attaching natural ligaments to bone. Device 10 may be used in ligament replacement procedures to attach the respective ends of artificial or replacement ligament L_{A} to femur F and tibia T. Ligament L_{A} may be a synthetic or harvested graft ligament. In fact device 10 may be a ligament replacement system that includes artificial ligament L_{A}. In this case, ligament L_{A} may be of similar structure and composition as patch 12. For instance, ligament L_{A} may be formed of a flexible surgical grade woven material. The woven material may be woven of any surgical grade fibers including metal fibers, plastic fibers, synthetic fibers such as Kevlar®, rayon, acetate, and polyester or mixtures thereof. Alternatively, ligament L_{A} may be formed of a woven material different from that of patch 12, such that ligament L_{A} has a resiliency mimicking that of the natural ligament while patch 12 has strength and rigidity to resist deformation and tearing when attached ligament L_{A} pulls on patch 12 during knee movement.

Ligament L_{A} may have a smooth outer layer or coating to prevent or minimize irritation of surrounding tissues. The outer layer may be comprised of hydrogel woven with the fibers of the woven material forming ligament L_{A}. In this ligament replacement system, each end of ligament L_{A} would be coupled to a patch, such as patch 12 described above, for use in attached the ends to respective femur F and tibia T. The ends of ligament L_{A} may be attached to patch 12 using any means including sutures, staples or other attachment device. Alternatively, ends of L_{A} may be formed integrally with patch 12. For instance, the woven material of ligament L_{A} may be woven together at its ends with the woven material of patch 12.

Turning now to FIGS. 4A and 4B, another embodiment of the present invention is shown. Attachment device 110 includes patch 112 and reinforcing patch 28. Patch 112 is substantially the same as patch 12 of device 10 (FIGS. 1-2A). Accordingly, patch 112 may be similarly secured to tibia T, and ligament end L_{E} may be secured to patch 112. Reinforcing patch 28 is configured to fit atop patch 112 and over ligament L thereby sandwiching ligament end L_{E} between patch 112 and reinforcing patch 28. Similar to patch 12, both patch 112 and reinforcing patch 28 are formed of a material that permits the attachment of ligament fastener 26 thereto. The material of patch 112 and reinforcing patch 28 may be a weave of metal fibers, plastic fibers, and/or synthetic fibers such as Kevlar®, acetate, rayon and polyester. Alternatively, the material of patch 112 and reinforcing patch 28 may be in the form of a molded lattice or porous fabric.

Patch 112 and reinforcing patch 28 are adapted to receive bone fasteners 20, which extend through both reinforcing patch 28 and patch 112 to attach device 110 to bone. For example, patch 112 and reinforcing patch 28 may include bone fastener openings (not shown) through which bone fasteners 20 may extend. Bone fastener openings of reinforcing patch 28 may be aligned with bone fastener openings of patch 112 to facilitate fixation of device 110 to tibia T with bone fasteners 20. Bone fastener openings of both patch 112 and reinforcing patch 28 may be reinforced as described above with respect to patch 12 to form reinforced area 122 around bone fastener openings. Alternatively, fasteners 20 may be used to secure patch 112 to bone, while reinforcing patch 28 may be fixed to patch 112 using fasteners (not shown), such as sutures, staples or clips. The fasteners may extend through ligament end L_{E} to secure both ligament end L_{E} and reinforcing patch 28 to patch 112. Reinforcing patch 28 reinforces the fixation of ligament end L_{E} to tibia T by compressing and holding ligament end L_{E} between patch 112 and reinforcing patch 28.

Reinforcing patch 28 includes an interior ligament engaging face (not shown) and an exterior face 30. Interior ligament face may exhibit a mesh structure, which may help grip and hold ligament end L_{E} to further secure the fixation of ligament end L_{E} to tibia T. Exterior face 30 of reinforcing patch 28 may include a smooth coating (not shown) to prevent or minimize irritation of surrounding tissues. The smooth coating of exterior face 30 may be formed of hydrogel or ceramics.

Turning to FIGS. 5A-5D, the present invention also contemplates an embodiment that may be used to repair a centralized tear in a ligament; that is, a tear in the ligament at a position between the two ends such that the ends remain attached to bone. As illustrated in FIG. SA, ligament L has suffered a centralized tear leaving loose ligament ends L₁ and L₂. Attachment device 210 generally includes first patch 34 and second patch 36. Each of first and second patches 34, 36 are formed of material similar to those described above with respect to patch 12. In other words, first and second patches may be formed of woven metal fibers, plastic fibers and/or synthetic fibers. Alternatively, first and second patches may be formed of material such as a molded lattice or porous fabric. Each of first and second patches include interior face 35 and exterior face 37.

As shown in FIG. 5B, first patch 34 is positioned on one side of ligament L between femur F and ligament L. Ligament ends L₁, L₂ are pulled together and attached to first patch 34 using ligament fasteners 26, such as sutures, staples, or clips (FIG. SC). Second patch 36 is placed over ligament ends L₁, L₂ and atop first patch 34 thereby positioning ligament ends L₁, L₂ between interior faces 35 of first and second patches 34, 36.

Interior face 35 of each of first and second patches 34, 36 may include a ligament growth layer or coating (not shown). The ligament growth layer may include materials designed to induce the fusion of ligament ends L₁, L₂. For example, ligament growth layer may include growth factors, ligament cells, antibiotics, steroids, and other biologics that may encourage fusion of ligament ends L₁, L₂. The ligament growth layer may also include a ligament growth scaffold to provide a matrix for the growth and fusion of ligament ends L₁, L₂.

Exterior face 37 of each of first and second patches 34, 36 may include a smooth coating or layer to minimize interference by surrounding tissues during the movement of the ligament. Exterior face (not shown) of first patch 34, particularly, may have a smooth coating upon which femur F can glide. As patch 34 moves with the stretching, contracting, and other movement of ligament L, femur F may glide along the smooth coating such that the interaction between femur F and first patch 34 does not interfere with the movements of ligament L. Exterior face 37 of second patch 36 may also have a smooth surface upon which surrounding soft tissues can glide, thereby preventing or minimizing irritation of surrounding tissues. For example, the smooth surface may be formed of hydrogels or ceramics.

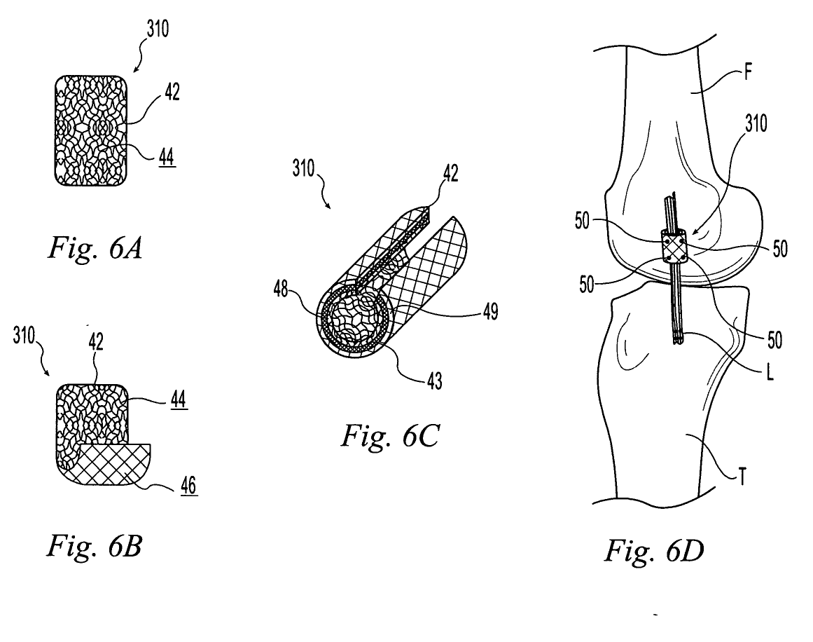

Referring now to FIGS. 6A-6D, attachment device 310 is shown. Device 310 includes patch 42 formed of a flexible material 43. Patch 42 includes interior ligament face 44 and opposite exterior face 46. Patch 42 is deformable to fold or roll over to wrap around two loose ends of tom ligament L (FIG. 6D). Accordingly, material 43 may be a woven material or fabric formed of metal fibers, plastic fibers, or other synthetic fibers including Kevlar® rayon, acetate, and polyester. The material of patch 42 is structured to provide deformability and to receive ligament fasteners 50, but stiff enough to maintain folded shape and support torn ligament ends of ligament L. Interior ligament face 44 of patch 42 may include ligament growth layer 48. Ligament growth layer 48 may comprise a scaffold for the growth and development of ligament L and to facilitate the fusion of the two ligament ends. Ligament growth layer 48 may also include biologics such as growth factors, ligament cells, or other materials that may induce the fusion of the two ends of ligament L.

Exterior face 46 of patch 42 may include a smooth coating or layer 49 upon which surrounding bone and soft tissue can glide, thereby minimizing the interference by surrounding tissues of the movement of ligament L.

As illustrated in FIG. 6D, patch 42 may be wrapped around the loose ends of ligament L, such that interior ligament face 44 surrounds the loose ends of ligament L. Ligament fasteners (not shown) may be used to attach the loose ends of ligament L to patch 42. Patch 42 may then be rolled or folded around ligament L. Fasteners 50 may be used to attach loose ends to folded/rolled patch 42 and hold patch 42 in its folded or rolled configuration. Fasteners 50 may include, for example, sutures, staples, or other fasteners. Ligament growth layer 48 facilitates the fusion of loose ends of ligament L to one another by providing biologics and scaffolding for the growth and development of ligament L. Patch 42 may be formed of biodegradable materials such that patch 42 degrades when fusion of loose ends of ligament L is achieved.

It should be understood that the attachment device of the present invention may have various sizes and shapes. Furthermore, the attachment device is not limited to use with ligaments of the knee, but rather is useful for the attachment or repair of various soft tissues, such as ligaments and tendons, in various locations of the body, such as ankle, hip, wrist, and others.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A device for attaching an end of a ligament to a bone, the device comprising:
a patch having a bone engaging face and a ligament engaging face, said patch adapted to be fastened to a surface of the bone such that said bone engaging face bears against the surface of the bone; and
a ligament fastener, said ligament engaging face adapted to receive said ligament fastener to attach the end of the ligament to said patch.

2. The device of claim 1 wherein said patch includes at least one opening, said at least one opening adapted to receive a bone fastener for fastening said patch to the bone.

3. The device of claim 2 wherein said patch is reinforced about a perimeter of said at least one opening.

4. The device of claim 1 wherein said bone engaging face comprises a bone engaging layer, said bone engaging layer including growth factors.

5. The device of claim 1 wherein said bone engaging face comprises a bone engaging layer, said bone engaging layer including a bone ingrowth scaffolding.

6. The device of claim 1 wherein said ligament fastener comprises a suture.

7. The device of claim 1 wherein said patch is formed of a woven material.

8. The device of claim 7 wherein said woven material comprises a metal.

9. The device of claim 7 wherein said woven material comprises synthetic fibers.

10. The device of claim 1 further including a reinforcing patch, said reinforcing patch positionable atop and attachable to said patch, such that the end of the ligament is positioned between said patch and said reinforcing patch.

11. A device for re-attaching two ends of a torn ligament to one another, the device comprising:
a patch having an exterior face and an interior face; and
at least one ligament fastener, said patch adapted to receive said least one ligament fastener to attach the two ends of the ligament to said interior face;
wherein said patch formable to wrap around the two ends of the ligament.

12. The device of claim 11 wherein said interior face of said patch includes a ligament growth layer, said ligament growth layer comprising a scaffolding structure.

13. The device of claim 11 wherein said interior face of said patch includes a ligament growth layer, said ligament growth layer including growth factors.

14. The device of claim 11 wherein said patch is formed of a woven material.

15. The device of claim 12 wherein said scaffolding structure comprises a hydrogel.

16. The device of claim 11 wherein said patch is biodegradable.

17. The device of claim 11 wherein said at least one ligament fastener is a suture.

18. A device for re-uniting two ends of a torn ligament, said device comprising:
a first patch and a second patch, each of said first and second patches having an interior ligament face and an exterior face, said first patch positionable adjacent a first side of the ends of the ligament, said woven material adapted to receive a ligament fastener for attaching the ends of the ligament to said interior ligament face, said second patch positionable atop said first patch and adjacent an opposite second side of the ends of the ligament such that the ends of the ligament are positioned between said interior faces of said first and second patches, said second patch attachable to said first patch.

19. The device of claim 18 wherein each of said first and second patches comprises a woven material.

20. The device of claim 18 wherein said interior face of at least one of said first and second patches comprises a biologic material.

21. A ligament repair system for replacing a damaged ligament, said ligament replacement system comprising:
a replacement ligament having a first bone engaging end and a second bone engaging end; and
first and second patches respectively coupled to said first and second bone engaging ends, each of said first and second patches having a bone engaging face and a ligament engaging face, each of said first and second patches adapted to be fastened to a surface of a respective bone such that said bone engaging face bears against the surface of the respective bone; and
a ligament fastener, said ligament engaging face adapted to receive said ligament fastener to attach said first and second bone engaging ends of the ligament to said first and second patches respectively.

22. The device of claim 21 wherein each of said first and second patches is formed of a first woven material and said replacement ligament is formed of a second woven material.

23. The device of claim 21 wherein said first woven material is different from said second woven material.

24. The device of claim 22 wherein said first woven material is woven into said second woven material to thereby integrally couple said first and second patches to said first and second bone engaging ends, respectively.

25. The device of claim 22 wherein said replacement ligament includes a hydrogel coating woven into said second woven material.

26. The device of claim 21 wherein said bone engaging face comprises a bone engaging layer, said bone engaging layer including growth factors.

27. The device of claim 21 wherein said bone engaging face comprises a bone engaging layer, said bone engaging layer including a bone ingrowth scaffolding.
